(19) United States
(12) Patent Application Publication
(10) Pub. No.: US 2007/0043422 A1
(43) Pub. Date: Feb. 22, 2007

(54) DELIVERY SYSTEM AND METHOD OF USE FOR ACCURATE POSITIONING OF A DEVICE IN A BIFURCATION

(75) Inventors: Ascher Shmulewitz, Tel Aviv (IL); Menashe Yacoby, Shoham (IL); Gil Noar, Ramat-Hasharon (IL); Raz Bar-On, Tel Adashim (IL)

Correspondence Address: RISSMAN JOBSE HENDRICKS & OLIVERIO, LLP, ONE STATE STREET, SUITE 800, BOSTON, MA, 02109, US

(73) Assignee: Cappella, Inc., Auburndale, MA (US)

(21) Appl. No.: 11/465,562

(22) Filed: Aug. 18, 2006

Related U.S. Application Data
(60) Provisional application No. 60/709,154, filed on Aug. 18, 2005.

(51) Int. Cl. A61F 2/06

(52) U.S. Cl. 623/1.11

(57) ABSTRACT

A delivery system for delivering a stent to a bifurcation in a vessel. The delivery system includes two guidewire lumens for directed the stent through the anatomy to the bifurcation. A main branch guidewire lumen has entry and exit ports that are positioned in order to limit how far the delivery system will be allowed into a side branch vessel. The orientation of the main branch guidewire lumen causes the delivery system to bind on the main branch guidewire as the system is positioned in the side branch. The location of the main branch guidewire lumen openings will physically limit the distance into the side branch that the stent will be placed.

### RELATED APPLICATIONS

This application is a non-provisional application of provisional patent application 60/709,154, filed Aug. 18, 2005 entitled “Delivery System And Method Of Use For Accurate Positioning Of a Device In a Bifurcation,” the entire contents of which is incorporated by reference.

### FIELD OF THE INVENTION

The present invention pertains to a system and method for delivering an implant or device to a bifurcation or ostium site in a body lumen, e.g., a blood vessel. More particularly, this invention pertains to a delivery system for a self-expandable implant that uses two guidewires to facilitate positioning of the implant at the desired location in the body lumen.

### BACKGROUND OF THE INVENTION

It is known to repair diseased vessels by placing a prosthesis, e.g., a stent, at the diseased location. Repairing a vessel with a diseased bifurcation area is particularly challenging because the prosthesis must overlay the entire diseased area at the bifurcation, yet not itself compromise blood flow. If the prosthesis does not overlay the entire circumference of the ostium to the diseased portion, the prosthesis may fail to completely repair the bifurcated vessel. Where the prosthesis overlays the entire circumference of the ostium to the diseased portion, yet extends into the junction comprising the bifurcation, the diseased area is repaired, however, blood flow may be compromised. Moreover, by extending into the junction comprising the bifurcation, the prosthesis may block access to portions of the bifurcated vessel that require further interventional procedures. For at least these reasons, accurate positioning of a repair device is critical for successful treatment of disease at a bifurcation.

### SUMMARY OF THE INVENTION

A delivery system is composed of a tubular catheter that is guided on two guidewires that are placed, one in the main branch, and one in the side branch of the intended bifurcation vessel. The delivery system is guided on the two guidewires until it is bounded by the two guidewires from any further distal movement into the side branch. At this point, the delivery system is positioned at a predetermined or known location relative to the bifurcation anatomy. The delivery system may then be pulled back a predetermined or known distance, if necessary, before the treatment is delivered.

The system, in one embodiment, has two separate lumens, one for each guidewire, that may run at the center of the tube, or adjacent to the center, for the full length of the tube or for a portion of the tube. One lumen may start from the distal end of the system and continue to any location along the tube where the lumen will have an opening for the guidewire to exit the tube. The second lumen may start at the proximal end of the system, or at any point along the tube, and continue for any length before the distal end of the system where the second lumen will have an opening for the guidewire to exit.

In one embodiment, a delivery system comprises a catheter having a tubular shell with a proximal end and a distal end; a first lumen disposed in the catheter, the first lumen having a distal opening located at the distal end of the catheter and a proximal opening located on the tubular shell proximal to the distal end of the tubular shell; a second lumen disposed in the catheter, the second lumen having a distal opening located on the tubular shell a predetermined distance from the distal end of the catheter and a proximal opening located on the tubular shell proximal to the second lumen distal opening; and a device positioned near the distal end of the catheter, wherein the predetermined distance is greater than a length of the device such that the second lumen distal opening is located proximally with respect to a proximal end of the device.

In accordance with another embodiment of the present invention, a delivery system comprises a catheter having a tubular shell with a proximal end and a distal end; a first lumen disposed in the catheter, the first lumen having a distal opening located at the distal end of the catheter; a second lumen disposed in the catheter, the second lumen having a distal opening located on the tubular shell a predetermined distance from the distal end of the catheter; and a device positioned near the distal end of the catheter, wherein the predetermined distance is greater than a length of the device such that the second lumen distal opening is located proximally with respect to a proximal end of the device.

A method of delivering a device to a location in a vessel system of a patient, the device positioned on a catheter having a tubular shell with a proximal end and a distal end, a first lumen disposed in the catheter, the first lumen having a distal opening located at the distal end of the catheter, a second lumen disposed in the catheter, the second lumen having a distal opening located on the tubular shell a predetermined distance from the distal end of the catheter, wherein the predetermined distance is greater than a length of the device such that the second lumen distal opening is located proximally with respect to a proximal end of the mounted device and no portion of the second lumen passes through a lumen of the mounted device, the method comprising: inserting a first guidewire into a first vessel of the vessel system; inserting a second guidewire into a second vessel of the vessel system; inserting a proximal end of the first guidewire through the first lumen distal opening and into the first lumen; inserting the second guidewire into the second lumen distal opening and into the second lumen; inserting the catheter into the patient and advancing the catheter along the first guidewire into the first vessel; determining whether the second guidewire is binding on the catheter; and if it is determined that the second guidewire is binding on the catheter, terminating advancement of the catheter into the first vessel.

In another embodiment, a delivery system comprises a tubular catheter having a proximal end and a distal end; treating means, for applying treatment to a vessel, positioned near the distal end of the catheter, the treating means comprising distal and proximal ends; a first lumen extending from a first opening at the distal end of the catheter to a second opening, the second opening of the first lumen proximally located with respect to the distal end of the catheter; and a second lumen extending from a third opening in the catheter to a fourth opening in the catheter, the third opening proximally located a predetermined distance from the distal end of the catheter.

### BRIEF DESCRIPTION OF THE DRAWINGS

The above and further advantages of the invention may be better understood by referring to the following description in conjunction with the accompanying drawings in which:

FIG. 1 is a drawing of a delivery system according to one embodiment of the present invention;

FIG. 2 is a drawing of a delivery system according to another embodiment of the present invention;

FIG. 3 is a drawing of a vessel system;

FIGS. 4A-4C represent placement of a delivery system in the vessel system of FIG. 3;

FIG. 5 is a drawing of a delivery system according to yet another embodiment of the present invention; and

FIG. 6 is a flowchart of a method according to one embodiment of the present invention.

### DETAILED DESCRIPTION

To overcome the problem of inaccurate positioning commonly shared by available delivery systems, an improved delivery system is hereby disclosed that facilitates the accurate positioning of a tubular device in a bifurcated vessel.

The invention is herein described, by way of example only, with reference to the accompanying drawings. It is stressed that the particulars shown are by way of example and for purposes of illustrative discussion of the various embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Prior to explaining at least one embodiment of the present invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

It is appreciated that certain features of the invention, which are, for the sake of clarity, described in the context of separate embodiments, may also be provided in combination in a single embodiment. Conversely, various features of the invention, which are, for brevity, described in the context of a single embodiment, may also be provided separately or in any suitable subcombination.

An improved catheter assembly and method of use are provided for treating bifurcated vessels. A delivery system in which intravascular treatment, such as, but not limited to, drug delivery device placement, prosthesis placement, balloon angioplasty, etc., can be applied or positioned accurately at a bifurcation side branch is described. The risk of device misplacement and/or excessive treatment duration is minimized due to the accurate positioning characteristics of the delivery system.

The system is composed of a tubular catheter that is guided on two guidewires that are placed, respectively, one in the main branch, and one in the side branch, of the target bifurcation vessel. The system, in one embodiment, has two separate lumens, one for each guidewire, that may run at the center of the tube, or adjacent to the tube, for the full length of the lumen or for a discrete section of the lumen. One lumen may start from the distal end of the system and continue to any location along the tube where it will have an opening for the guidewire to exit the tube. The second lumen may start at the proximal end of the system, or at any point along the tube, and continue for any length before reaching the distal end of the system where the second lumen will have an opening for the guidewire to exit.

The delivery system is guided on the two guidewires until it is bounded or prevented from any further distal movement by the bifurcation anatomy. At this point the delivery system is positioned at a predetermined location relative to the bifurcation anatomy. The delivery system may then be pulled back a certain distance, if necessary, before the treatment is delivered to the bifurcation. After the treatment is delivered, the system can be withdrawn while leaving each guidewire in its respective vessel for providing additional treatment or the guidewires can be withdrawn.

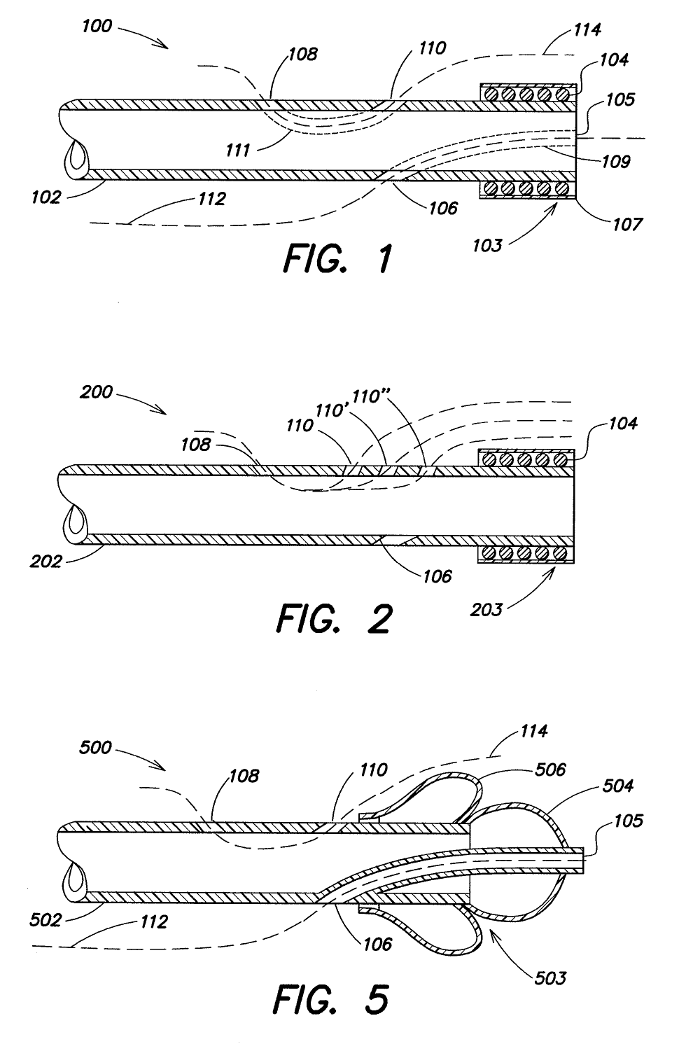

One embodiment of the delivery system will now be described with reference to FIG. 1. As shown, a delivery system 100 includes a catheter 102 having a device 104 mounted near a distal end 103 thereof. The device 104 does not have to placed directly at the distal end 103 of the catheter and, in most applications, will be “set back” from the distal end 103. The device 104 may be, for example, a drug delivery device, a balloon for balloon angioplasty, or a stent. In the instance where the device 104 is a stent, the stent may be of a self-expanding type, e.g., made from a shape-memory material such as Nitinol, or self-expanding due to energy stored in resiliently biased portions of the device, e.g., spring-like connectors, or a balloon-expandable type of device. Where the device 104 is a self-expanding stent, the catheter 102 may comprise a slideable sheath 107 that operates to constrain the stent on the catheter in a compressed state and, when pulled aside, allows the stent to expand to its non-constrained shape. One of ordinary skill in the art will understand that there are other mechanisms for releasably mounting a stent on the catheter 102 that may be used in one or more of the embodiments of the present invention.

The catheter 102 is, in one embodiment, made of kink resistant extruded polymer tubing. Polymers such as nylon, PEBAX, polyethylene, or polyester may be used. Alternatively, thermoset polymers such as polyimide or braid reinforced polyimide may be used. One of ordinary skill in the art will understand that there are other materials from which the catheter 102 may be made.

A side branch lumen 109, shown in dotted line, has a side branch distal exit port 105 provided at the distal end 103 of the catheter 102 and a side branch proximal exit port 106 provided in the catheter 102 at a location proximal to the distal end 103 of the catheter 102. A main branch lumen 111 has a main branch proximal exit port 108 and a main branch distal exit port 110 provided in the side wall of the catheter 102. In operation, as will be described below, a side branch guidewire will generally follow a side branch guidewire path 112 (as shown by the dashed line) from the side branch distal exit port 105 at the distal end 103 of the catheter 102 through to the side branch guidewire exit port 106. A main branch guidewire will generally follow a main branch guidewire path 114 through the main branch distal exit port 110 and out the main branch proximal exit port 108.

It should be noted that the relative positions of the side branch exit port 106, the main branch proximal exit port 108, and the main branch distal exit port 110, are provided for explanatory purposes only and not meant to be limiting. One of ordinary skill in the art would understand that, in accordance with the teachings of the present disclosure, the specific placement of these ports, e.g., a distance between them, the locations around a circumference of the catheter 102, etc., will depend on other system design factors or considerations. Further, while the positioning of the ports 106, 108, 110 is in a configuration known in the art as a rapid exchange (RX) configuration, these ports 106, 108, 110 could be configured to provide an over the wire (OTW) system as well. In an OTW system the main branch proximal exit port 108 and the side branch exit port 106 are located at the proximal end of the catheter 102 instead of in the side wall. Still further, a main branch guidewire lumen and a side branch guidewire lumen may be separately provided within the catheter 102 to direct the main branch guidewire and side branch guidewire, respectively.

It will be noted that any lumens within which the guidewires would travel are not further shown in order to make the figures easier to understand. One of ordinary skill in the art will understand how guidewire lumens may be provided within the catheter 102 in order to achieve the functionality and teachings of the present invention. The lumens could be coaxial, or side-by-side, or the catheter 102 may have a bi-lumen construction.

In an alternate embodiment, as shown in FIG. 2, a delivery system 200 comprises a catheter 202 that has a plurality of main branch distal exit ports 110, 110′ and 110″. By providing multiple main branch distal exit ports 110 on the catheter 202, this embodiment of the present invention provides for placement of the device 104 at various depths or distances, as will be understood by the description below. The multiple main branch distal exit ports 110, 110′ and 110″ may either comprise removable portions in order to access the exit ports or a system where unused exit ports are covered or sealed off prior to use. Further, markings on the exterior of the catheter 202 may be provided to indicate a device placement depth corresponding to each main branch distal exit port, i.e., a distance to a distal end 203 of the catheter 202.

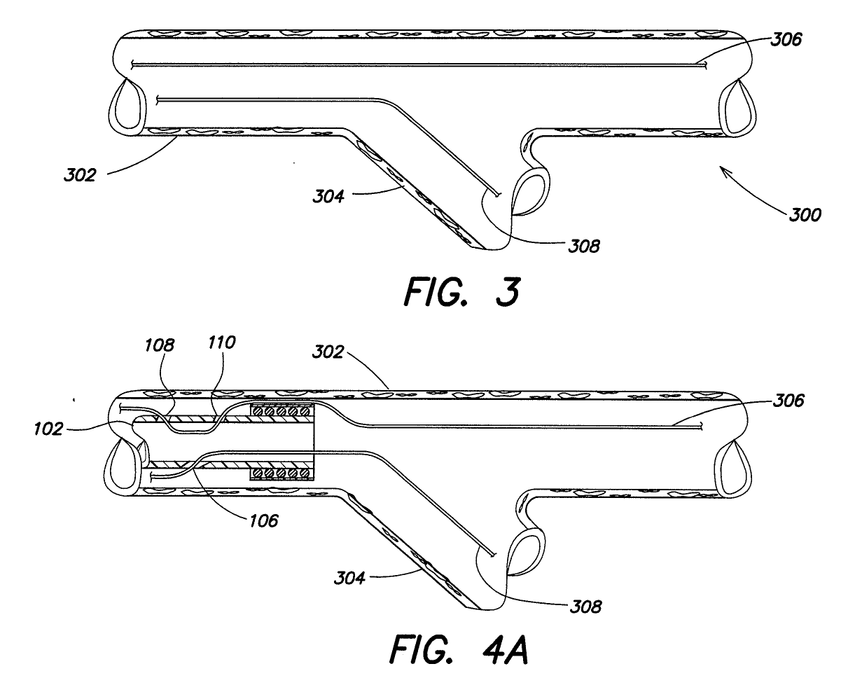

Referring now to FIG. 3, a vessel system 300 within a patient includes a main branch vessel 302 and a side branch vessel 304. In operation of the embodiments of the present invention, a main branch guidewire 306 is positioned in the main branch vessel 302 while a side branch guidewire 308 is positioned, via the main branch vessel 302, into the side branch vessel 304. The positioning or placement of a guidewire in a patient's lumen is well known in the art.

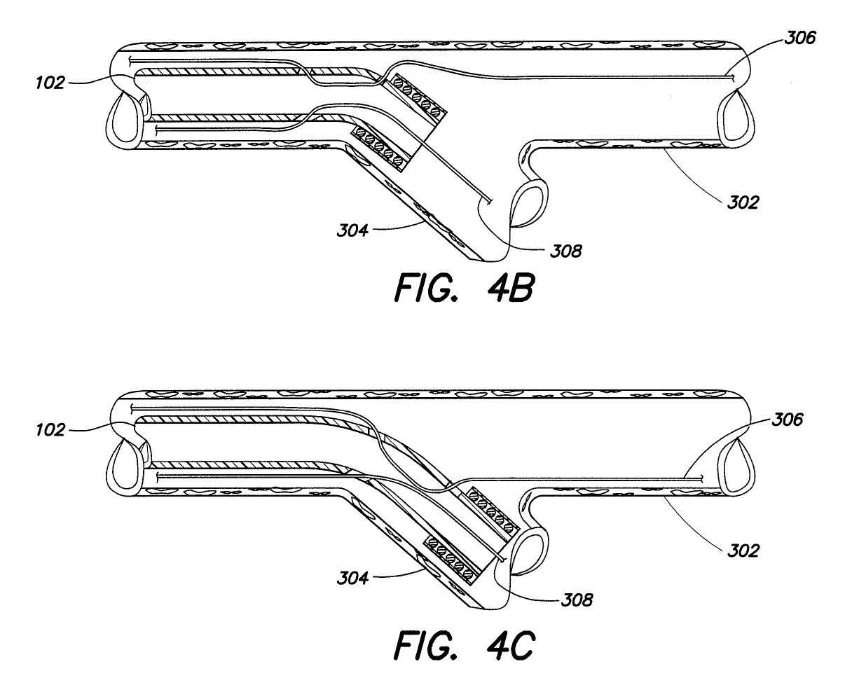

The positioning of the catheter 102 will now be described with respect to FIGS. 4A-4C. The following description refers to the catheter 102 for ease of explanation although the process is applicable to any embodiment described herein. As shown in FIG. 4A, the catheter 102 is introduced into the vessel system of the patient after being mounted to follow each of the main branch guidewire 306 and the side branch guidewire 308. In operation, a proximal end of the main branch guidewire 306 is fed into the main branch distal exit port 110 and exits through the main branch proximal exit port 108. A proximal end of the side branch guidewire 308 is fed into the distal end 103 of the catheter 102 and exits through the side branch exit port 106.

The catheter 102 is urged through the vessel system 300 and follows the side branch guidewire 308. The movement of a catheter within a patient's vessel system is well known in the art and will not be discussed in detail here. As shown in FIG. 4B, the catheter 102 follows the side branch guidewire 308 into the side branch vessel 304. As the catheter 102 turns, or is directed, into the side branch 304, the main branch guidewire 306 will begin to bind on the catheter 102. This binding or resistance is detected by the operator of the delivery system and is used to position the distal end of the catheter 102 at the desired location within the side branch 304. As can be seen, a distance from the distal end 103 of the catheter 102 to the main branch distal exit port 110 will determine how far into the side branch 304 the catheter 102 and, therefore, the device 104, will be positioned.

The binding of the main branch guidewire 306 may be detected by the operator as a resistance to further insertion that is felt in response to attempting to advance the catheter 102. Alternatively, via imaging equipment as known in the art, changes in the position of the guidewire and/or the distal end of the catheter 102 may be visualized.

One or more of the catheter 102, device 104, the main branch guidewire 306 and the side branch guidewire 308, may include radio-opaque markers (not shown) to permit the operator to visualize a position of the system within the vessel system 300 via fluoroscopy. For example, a Platinum/Iridium (Pt/Ir) alloy band may be attached or integrated into a component's construction as a marker. Thus, by viewing the positions of the markers and any changes in position, or lack thereof, as the catheter 102 is advanced, the operator can determine when the main branch guidewire 306 is binding on the catheter 102 and that the catheter 102 has been sufficiently advanced into the vessel system 300.

Once the distal end 103 of the catheter 102 is at the desired location, the device 104 is released or operated by mechanisms known to those skilled in the art depending upon the type of device 104, i.e., self-expanding stent, balloon expandable stent, or balloon angioplasty device, that is being delivered to the patient.

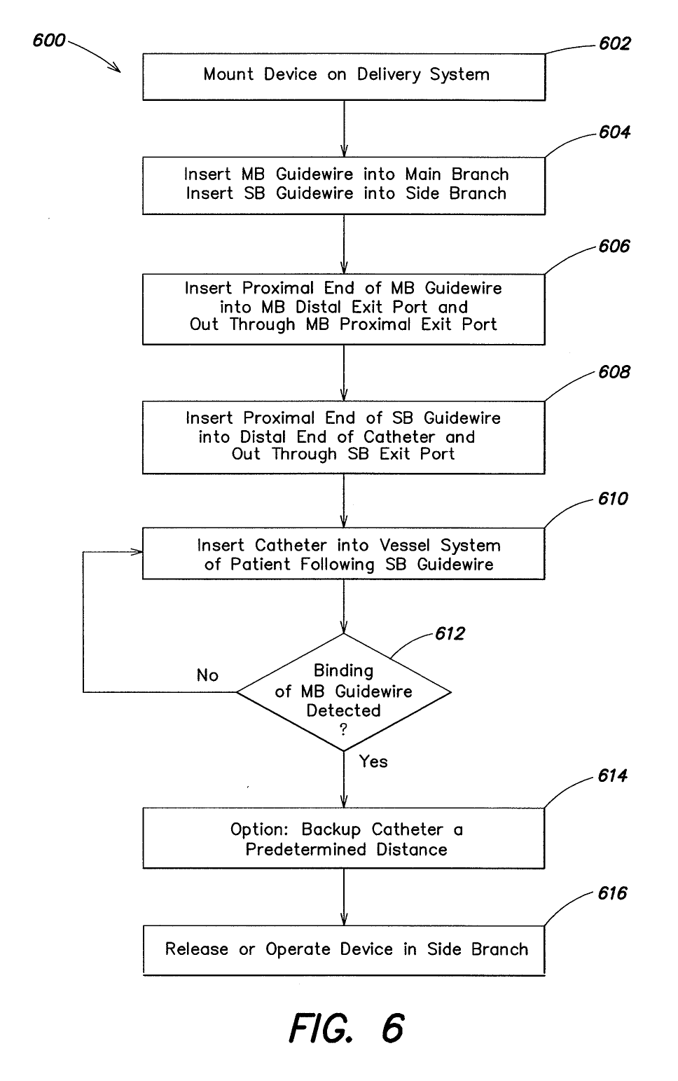

A method 600 of operation, according to one embodiment of the present invention, begins with mounting a device 104 on the catheter 102, step 602, as shown in FIG. 6. Next, step 604, the main branch guidewire 306 and the side branch guidewire 308 are inserted into the main branch vessel 306 and side branch vessel 304, respectively. The proximal end of the main branch guidewire 306 is inserted through the main branch distal exit port 110 and proximal exit port 108, step 606. The proximal end of the side branch guidewire 308 is inserted into the distal end 103 of the catheter 102 and exits the side branch exit port 106, step 608. At step 610, the catheter 102 is inserted into the patient and follows the side branch guidewire 308 into the side branch vessel 304. If the binding of the main branch guidewire 306 is not detected at step 612, advancement of the catheter 102 is continued. If binding or resistance is detected by the operator, then the advancement of the catheter 102 is stopped. Optionally, the catheter 102 then can be backed-off a predetermined distance, step 614. Once binding is detected and advancement is stopped, with or without having backed-off, the device is released or used to apply treatment at the desired location at step 616.

In an alternate embodiment as shown in FIG. 5, a delivery system 500 includes a catheter 502 with a semi-compliant balloon 504 located at a distal end 503 and a compliant balloon 506 located proximally relative to the semi-compliant balloon 504. Similar to the delivery systems as shown in FIGS. 1 and 2, the delivery system 500 includes the side branch distal and proximal exit ports 105,106 and the main branch proximal and distal exit ports 108,110. Alternatively, the delivery system 500 may have an OTW configuration. This system may be used for performing balloon angioplasty in one of the bifurcation branches.

The catheter 102 may be provided in various diameters and lengths to accommodate various sizes of patients and vessels. Further, versions of the catheter 102 may be made with various device placement depths as defined by the distance from the distal end of the catheter 102 to the main branch distal exit port 110.

Yet another aspect of the present invention is the use of the delivery system for targeting one of the bifurcation branches when a prosthesis is already present in the bifurcation, partially or fully covering the ostium of that branch.

An advantage of the described invention is that it may reduce overall treatment time by facilitating easier and faster positioning of the treatment at the intended site, thereby reducing patient exposure to X-ray radiation.

Another advantage of the described invention is that it reduces the chances of mis-positioning a bifurcation device due to X-ray imaging overlay.

Yet another advantage of the described invention is the ability to follow along a side branch guidewire that is not implemented so as to pass through a side of a stent being delivered to the patient. As described, the side branch guidewire passes through the central axis of the catheter and not through, for example, the interstitial spaces between the struts of the stent or similar device. This construction reduces the complexity of the system while, at the same time, providing for more precise delivery capabilities. Further, the risk of damage to the device is reduced by not having a guidewire pass through a narrow space or without having to open a large space in the side of the device in order to pass the guidewire.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the invention. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A delivery system, comprising:
a catheter having a tubular shell with a proximal end and a distal end;
a first lumen disposed in the catheter, the first lumen having a distal opening located at the distal end of the catheter and a proximal opening located on the tubular shell proximal to the distal end of the tubular shell;
a second lumen disposed in the catheter, the second lumen having a distal opening located on the tubular shell a predetermined distance from the distal end of the catheter and a proximal opening located on the tubular shell proximal to the second lumen distal opening; and
a device positioned near the distal end of the catheter,
wherein the predetermined distance is greater than a length of the device such that the second lumen distal opening is located proximally with respect to a proximal end of the device.

2. The delivery system of claim 1, wherein the first and second lumens are not in fluid contact with one another.

3. The delivery system of claim 1, wherein the device is releasably positioned on the catheter.

4. The delivery system of claim 1, wherein no portion of the second lumen passes through a lumen of the mounted device.

5. The delivery system of claim 1, wherein the device comprises a stent.

6. The delivery system of claim 1, wherein the device comprises an angioplasty balloon.

7. A delivery system, comprising:
a catheter having a tubular shell with a proximal end and a distal end;
a first lumen disposed in the catheter, the first lumen having a distal opening located at the distal end of the catheter;
a second lumen disposed in the catheter, the second lumen having a distal opening located on the tubular shell a predetermined distance from the distal end of the catheter; and
a device positioned near the distal end of the catheter,
wherein the predetermined distance is greater than a length of the device such that the second lumen distal opening is located proximally with respect to a proximal end of the device.

8. The delivery system of claim 7, wherein:
the first lumen further comprises a proximal opening located on the tubular shell proximal to the distal end of the tubular shell.

9. The delivery system of claim 7, wherein the device comprises a stent.

10. The delivery system of claim 7, wherein the first and second lumens are separate from one another.

11. The delivery system of claim 7, wherein the device comprises an angioplasty device.

12. The delivery system of claim 7, wherein the device is releasably positioned on the catheter.

13. The delivery system of claim 7, wherein:
the second lumen further comprises a proximal opening located on the tubular shell proximal to the second lumen distal opening.

14. The delivery system of claim 13, wherein no portion of the second lumen passes through a lumen of the mounted device.

15. A method of delivering a device to a location in a vessel system of a patient, the device positioned on a catheter having a tubular shell with a proximal end and a distal end, a first lumen disposed in the catheter, the first lumen having a distal opening located at the distal end of the catheter, a second lumen disposed in the catheter, the second lumen having a distal opening located on the tubular shell a predetermined distance from the distal end of the catheter, wherein the predetermined distance is greater than a length of the device such that the second lumen distal opening is located proximally with respect to a proximal end of the mounted device and no portion of the second lumen passes through a lumen of the mounted device, the method comprising:
inserting a first guidewire into a first vessel of the vessel system;
inserting a second guidewire into a second vessel of the vessel system;
inserting a proximal end of the first guidewire through the first lumen distal opening and into the first lumen;
inserting the second guidewire into the second lumen distal opening and into the second lumen;
inserting the catheter into the patient and advancing the catheter along the first guidewire into the first vessel;
determining whether the second guidewire is binding on the catheter; and
if it is determined that the second guidewire is binding on the catheter, terminating advancement of the catheter into the first vessel.

16. The method of claim 15, wherein the device is releasably positioned on the catheter, the method further comprising:
releasing the device from the catheter.

17. The method of claim 16, further comprising:
backing the catheter a predetermined distance out of the vessel system prior to releasing the device.

18. The method of claim 15, wherein determining whether the second guidewire is binding on the catheter comprises:
detecting resistance to advancement of the catheter into the first vessel.

19. The method of claim 18, wherein determining whether the second guidewire is binding on the catheter further comprises:
visually detecting resistance to advancement of the catheter into the first vessel.

20. The method of claim 15, wherein the device comprises an angioplasty device.

21. The method of claim 20, further comprising:
operating the angioplasty device within the vessel system.

22. A delivery system, comprising:
a tubular catheter having a proximal end and a distal end;
treating means, for applying treatment to a vessel, positioned near the distal end of the catheter, the treating means comprising distal and proximal ends;
a first lumen extending from a first opening at the distal end of the catheter to a second opening, the second opening of the first lumen proximally located with respect to the distal end of the catheter; and
a second lumen extending from a third opening in the catheter to a fourth opening in the catheter, the third opening proximally located a predetermined distance from the distal end of the catheter.

23. The system of claim 22, wherein the treating means comprise:
a self-expanding stent releasably mounted near the distal end of the catheter, the stent having proximal and distal ends.

24. The system of claim 23, wherein the predetermined distance is greater than a distance from the proximal end of the mounted stent to the distal end of the catheter.

25. The system of claim 24, further comprising:
means for holding the stent in a compressed state about the catheter.

26. The system of claim 22, wherein the treating means comprise:
an angioplastic balloon.
